(19) United States
(12) Patent Application Publication
(10) Pub. No.: US 2023/0330229 A1
(43) Pub. Date: Oct. 19, 2023

(54) METHODS FOR IMAGE-GUIDED RADIOTHERAPY

(71) Applicants: UNIVERSITE CLAUDE BERNARD LYON 1, Villeurbanne (FR); NH THERAGUIX, Meylan (FR); CENTRE GEORGES FRANCOIS LECLERC, Dijon (FR); The Brigham and Women's Hospital, Inc., Boston, MA (US)

(72) Inventors: François LUX, Lyon (FR); Olivier TILLEMENT, Fontaines Saint-Martin (FR); Géraldine LE DUC, Crolles (FR); Aurélien MEYZAUD, Lyon (FR); Magali ROUFFIAC, Dijon (FR); Céline MIRJOLET, Dijon (FR); Daniel CAGNEY, Boston, MA (US); Ross BERBECO, Boston, MA (US); Needa Virani, Brighton, MA (US)

(21) Appl. No.: 17/998,708

(22) PCT Filed: May 11, 2021
(86) PCT No.: PCT/EP2021/062502

§ 371 Date: Nov. 14, 2022

(30) Foreign Application Priority Data
May 15, 2020 (EP) 2030553.3

(51) Int. Cl. A61K 41/00; A61K 49/18; A61P 35/04; A61N 5/10

(52) U.S. Cl. CPC A61K 41/0038; A61K 49/1881; A61P 35/04; A61N 5/1049; A61N 5/1067; A61N 2005/1055; A61N 2005/1098

(57) ABSTRACT

The disclosure relates to methods for treating tumors. In particular, the disclosure relates to a method of treating a tumor by magnetic resonance image-guided radiation therapy in a subject in need thereof, said method comprising the steps of:
(i) administering an efficient amount of high-Z element containing nanoparticles having, contrast enhancement for magnetic resonance imaging and/or radiosensitizing properties for radiation therapy, in a subject in need thereof, and,
(ii) exposing said subject to magnetic resonance image-guided radiation therapy by means of a Magnetic Resonance Imaging Guided Linear Accelerator (MR-Linac),
   wherein said high-Z element containing nanoparticles are nanoparticles containing an element with an atomic Z number higher than 40, preferably higher than 50, and said nanoparticles have a mean hydrodynamic diameter below 20 nm, for example between 1 and 10 nm, preferably between 2 and 8 nm.

### TECHNICAL DOMAIN

The disclosure relates to methods for treating tumors. In particular, the disclosure relates to a method of treating a tumor by magnetic resonance image-guided radiation therapy in a subject in need thereof, said method comprising the steps of:
(i) administering an efficient amount of high-Z element containing nanoparticles having, contrast enhancement for magnetic resonance imaging and/or radiosensitizing properties for radiation therapy, in a subject in need thereof, and,
(ii) exposing said subject to magnetic resonance image-guided radiation therapy by means of a Magnetic Resonance Imaging Guided Linear Accelerator (MR-Linac),
   wherein said high-Z element containing nanoparticles are nanoparticles containing an element with an atomic Z number higher than 40, preferably higher than 50, and said nanoparticles have a mean hydrodynamic diameter below 20 nm, for example between 1 and 10 nm, preferably between 2 and 8 nm.

### BACKGROUND

The radiation therapy (also known as radiotherapy) is one of the most used anti-tumor strategies. More than half of all patients with cancer are treated with ionizing radiation (IR) alone or in combination with surgery or chemotherapy. Recent progresses realized in medical physics (with the development of low/high energy radiation, the implementation of mono-, hypo- or hyper-fractionation schedule and the diversification of dose rates used) and the development of innovative medical technologies (such as the 3D-conformational radiotherapy (3D-CRT), the intensity modulated radiation therapy (IMRT), the stereotactic radiosurgery (SRS) and the functional imaging) contribute to better deliver the efficient doses of radiation on tumors whilst sparing surrounding healthy tissues, which is the most usual side effect of radiation therapy.

Combining magnetic resonance (MR) scanner with a linear accelerator in a single machine is a recent development which could greatly improve the outcome of cancer radiotherapy. In particular, MR imaging offers the opportunity of improving tumor delineation especially for soft tissues cancers. The possibility to have MR imaging and treatment with ionizing radiations at the same time allows taking into account the spatial evolution of the tumor along time. In addition, real-time MR imaging can compensate the movement of the tumor and the organs at risk due to respiration.

However, one limitation for these emerging treatment protocols is that marketed contrast agents are difficult to use in that context. They have a short remanence in the tumors, which means that, in order to improve contrast of real-time imaging, one injection must be performed before each fraction of radiotherapy. In addition to generating an undue burden for patients and care givers this could be a safety issue since marketed contrast agents have been developed and validated for a limited exposure to patients.

Hence, there is a need to improve the protocol using MR image-guided radiation therapy.

The inventors made the unexpected finding that certain high-Z element containing nanoparticles provide suitable contrast within the tumors for MR imaging and/or radiosensitizing properties, for several days. This unexpectedly long remanence of such nanoparticles in the human tumors enable the inventor to design new therapeutic strategies combining magnetic resonance image-guided radiation therapy with the use of such high-Z element containing nanoparticles as contrast agent and/or radiosensitizing agent, wherein a single administration of the nanoparticles enables multiple fractions of magnetic resonance image-guided radiation therapy for the treatment of tumors in a subject in need thereof.

### BRIEF DESCRIPTION

Accordingly, the present disclosure relates to high-Z element containing nanoparticles, for use in a method of treating a tumor in a subject in need thereof, the method comprising
(i) administering an efficient amount of high-Z element containing nanoparticles having contrast enhancement for magnetic resonance imaging and/or radiosensitizing properties for radiation therapy, in a subject in need thereof, and,
(ii) exposing said subject to magnetic resonance image-guided radiation therapy by means of a Magnetic Resonance Imaging Guided Linear Accelerator (MR-Linac),
   wherein said high-Z element containing nanoparticles are nanoparticles containing an element with an atomic Z number higher than 40, preferably higher than 50, and wherein said nanoparticles have a mean hydrodynamic diameter 20 nm or less, for example between 1 and 10 nm, preferably between 2 and 8 nm.

In certain embodiments, said MR-Linac is preferably selected among MR-Linac with magnetic strength field of 0.5T, or lower strength field, for example 0.35T.

In certain embodiments, said nanoparticles comprise, as high-Z element, a rare earth metal, or a mixture of rare earth metals. For example, said nanoparticles may comprise, as high-Z element, gadolinium, bismuth, or a mixture thereof.

In certain embodiments, said nanoparticles comprise chelates of high-Z element, for example chelates of rare earth elements. Typically, said nanoparticles comprise
(i) polyorganosiloxane,
(ii) chelates covalently bound to said polyorganosiloxane,
(iii) high-Z elements complexed by the chelates.

In specific embodiments, said nanoparticles comprise
(i) polyorganosiloxane with a silicon weight ratio of at least 8% of the total weight of the nanoparticle, preferably between 8% and 50%,
(ii) chelates covalently bound to said polyorganosiloxane, in a proportion comprising between 5 and 100, preferably between 5 and 20 per nanoparticle, and,
(iii) high-Z elements complexed to the chelates.

In certain embodiments, said nanoparticles comprises chelates for complexing the high-Z elements, obtained by grafting one or more of the following cheating agents on said nanoparticles: DOTA, DTPA, EDTA, EGTA, BAPTA, NOTA, DOTAGA, and DTPABA, or their mixtures.

In particularly preferred embodiments, said nanoparticles are gadolinium-chelated polysiloxane nanoparticles, more preferably of the following formula

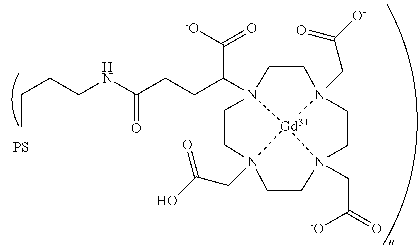

wherein PS is a matrix of polysiloxane, and,
n is comprised between 5 and 50, preferably 5 and 20, and wherein the hydrodynamic diameter is comprised between 1 and 10 nm, for example between 2 and 8 nm.

In certain embodiments, said method of treatment comprises a first tumor pre-filling step comprising administering an effective amount of said high-Z element containing nanoparticles as radiosensitizing agents in said subject in need thereof within a period between 2 and 10 days, preferably 2 and 7 days, prior to the first exposure to radiation therapy.

Advantageously, said subject may be exposed to at least one or more additional session of magnetic resonance image-guided radiation therapy, without further administration of a contrast agent for magnetic resonance imaging.

Typically, said subject is exposed to 2 or more sessions of magnetic resonance image-guided radiation therapy after a single administration of an efficient amount of said high-Z containing nanoparticles, for example between 2 and 7 sessions. In a more specific embodiment, said subject is exposed to 2 or more sessions of magnetic resonance image-guided radiation therapy within 5-7 days, typically with a minimum timeline of 2 or 3 days between each session.

In certain embodiments, the subject is exposed to a dose of ionizing radiations per session of magnetic resonance image-guided radiation therapy of about 3 Gy to about 20 Gy, and the total dose is administered preferably in a maximum of 10 fractions, for example in 1 to 10 fractions.

The tumor targeted by the method of the disclosure may be a solid tumor, preferably selected from
primary tumors of uterine cervix, rectum, lung, head and neck, prostate, colorectal, liver, and pancreas cancers and
bone metastases, typically undergoing intrafraction movements, such as sternal bones.

In certain embodiments, said nanoparticles are administered as an injectable solution at a concentration between 50 and 150 mg/mL, and preferably between 80 and 120 mg/mL, for example 100 mg/mL, preferably by intravenous injection. For example, a therapeutically effective amount administered for magnetic resonance image-guided radiation therapy is comprised between 50 mg/kg and 150 mg/kg, typically, between 80 and 120 mg/kg, for example 100 mg/kg.

### LEGENDS TO FIGURES

FIG. 1. MRI enhancement versus administered dose. Each point on the graph corresponds to an MRI enhancement value measured in a metastasis with longest diameter larger than 1 cm. MRI enhancements were found statistically different between each dose, the pooled 15-30 mg/kg dose, the pooled 50-75 mg/kg and 100 mg/kg.

FIG. 2. MRI enhancement versus AGuIX concentration. Each point on the graph corresponds to an MRI enhancement and AGuIX concentration value measured in a metastasis with longest diameter larger than 1 cm of patient #13. The black curve corresponds to a linear regression applied to the series of points. Dashed curved correspond to the 95% confidence bands.

FIG. 3. MRI enhancement one week after the administration of nanoparticles. Part of the signal enhancement map (color coded) of patient #13 is superimposed to the native 3D T₁-weighted images obtained 2 hours post i.v. injection (left side image) to the patient and one week later (right side image). The arrows are pointing the AGuIX-enhanced metastasis.

FIG. 4. MRI positive signal of Multihance (A) and AGuIX (B) using MRIdian from ViewRay.

FIG. 5. Intensity of signal for Multihance and AGuIX using MRIdian from ViewRay.

FIG. 6. Comparison of the MRI signal for AGuIX (A) and Bi-AGuIX (50/50) (B).

FIG. 7. quantification of mean signal intensity (C) using MRIdian from ViewRay.

FIG. 8. Imaging of subcutaneous NSCLC tumors after intravenous administration of AGuIX nanoparticles using MRIdian from ViewRay and TrueFISP (A) or T1-weighted (B) (Tumors are circled in white). Imaging of subcutaneous NSCLC tumors after intratumoral administration of AGuIX nanoparticles using MRIdian from ViewRay and TrueFISP sequence (C).

### DETAILED DESCRIPTION

The present disclosure follows in part from the surprising findings as shown by the inventors of a long remanence in human tumors of certain nanoparticles and their advantage for use as MR contrast agent and/or radiosensitizing agents in cancer treatment with multiple sessions of MR image-guided radiation therapies.

As used herein, the term “contrast agent” is intended to mean any product or composition used in medical imaging for the purpose of artificially increasing the contrast making it possible to visualize a particular anatomical structure (for example certain tissues or organs) or pathological anatomical structures (for example tumors) with respect to neighboring or non-pathological structures. The term “imaging agent” is intended to mean any product or composition used in medical imaging for the purpose of creating a signal making it possible to visualize a particular anatomical structure (for example certain tissues or organs) or pathological anatomical structures (for example tumors) with respect to neighboring or non-pathological structures (also referred hereafter as contrast enhancement). The principle of how the contrast or imaging agent operates depends on the imaging technique used.

Imaging may be performed using magnetic resonance imaging (MRI), computed tomography imaging, positron emission tomography imaging, or any combination thereof. As used herein, the term “MR contrast agent” refers to contrast agent which enables to enhance contrast in magnetic resonance imaging.

As used herein, the term “radiosensitizing” would be readily understood by one of ordinary skill in the art and generally refers to the process of increasing the sensitivity of the cancer cells to radiation therapy (e.g., photon radiation, electron radiation, proton radiation, heavy ion radiation, and the like).

The high-Z containing nanoparticles for use in the methods of treatment of the disclosure

Without being bound by any particular theory, it is believed that the advantageous effects of the methods of treatment of the present disclosure are linked in particular to two features of the nanoparticles:
they contain high-Z elements, typically complexes of high-Z cations, with radiosensitizing properties and/or contrast enhancement properties for MR imaging;
(ii) they have a small mean hydrodynamic diameter.

Said high-Z element as used herein is an element with an atomic Z number higher than 40, for example higher than 50.

In specific embodiments, said high-Z element is selected among the heavy metals, and more preferably, Au, Ag, Pt, Pd, Sn, Ta, Zr, Tb, Tm, Ce, Dy, Er, Eu, La, Nd, Pr, Lu, Yb, Bi, Hf, Ho, Pm, Sm, In, and Gd, and mixtures thereof.

The high-Z elements are preferably cationic elements, either comprised in the nanoparticles as oxide and/or chalcogenide or halide or as complexes with chelating agents, such as organic chelating agents.

The size distribution of the nanoparticles is, for example, measured using a commercial particle sizer, such as a Malvern Zetasizer Nano-S particle sizer based on PCS (Photon Correlation Spectroscopy).

For the purposes of the present disclosure, the term “mean hydrodynamic diameter” or “mean diameter” is intended to mean the harmonic mean of the diameters of the particles. A method for measuring this parameter is also described in standard ISO 13321:1996.

Nanoparticles with a mean hydrodynamic diameter for example below 20 nm, in particular between 1 and 10 nm, and even more preferably between 1 and 8 nm or for example between 2 and 8 nm, or typically around 5 nm, are suitable for the methods disclosed herein. In particular, they have been shown to provide excellent passive targeting in tumors, after intravenous injection, and a rapid renal elimination (and therefore low toxicity).

Preferably, said nanoparticles include at least 50% by weight of gadolinium (Gd), of dysprosium (Dy), of lutetium (Lu), of bismuth (Bi) or of holmium (Ho), or mixtures thereof, (relative to the total weight of high-Z elements in the nanoparticles), for example at least 50% by weight of gadolinium, as high-Z elements in the nanoparticles.

In a particularly preferred embodiment, said nanoparticle for use in the method of the present disclosure is a gadolinium-based nanoparticle.

In specific embodiments, said high-Z elements are cationic elements complexed with organic chelating agents, for example selected from chelating agents with carboxylic acid, amine, thiol, or phosphonate groups.

In preferred embodiment, the nanoparticles further comprise a biocompatible coating in addition to the high-Z element, and, optionally, the chelating agents. Agent suitable for such biocompatible includes without limitation biocompatible polymers, such as polyethylene glycol, polyethyleneoxide, polyacrylamide, biopolymers, polysaccharides, or polysiloxane.

In particular embodiments, the nanoparticles are chosen such that they have a relaxivity r1 per particle of between 50 and 5000 mM⁻¹·s⁻¹ (at 37° C. and 1.4 T) and/or a Gd weight ratio of at least 5%, for example between 5% and 30%.

In one specific embodiment, said nanoparticles with a very small hydrodynamic diameter, for example between 1 and 10 nm, preferably between 2 and 8 nm, are nanoparticles comprising chelates of high-Z elements, for example chelates of rare earth elements. In certain embodiments, said nanoparticles comprise chelates of gadolinium or bismuth.

In specific embodiments which may be combined with any of the previous embodiments, said high-Z element containing nanoparticles comprise
polyorganosiloxane,
chelating agents covalently bound to said polyorganosiloxane,
high-Z elements complexed by the chelating agents.

As used herein, the term “chelating agent” refers to one or more chemical moieties capable of complexing one or more metal ions.

Exemplary chelating agents include, but not limited to, 1,4,7-triazacyclononanetriacetic acid (NOTA), 1,4,7,10-tetraazacyclododecane-1,4,7,10-tetraacetic acid (DOTA), 1,4,7-triazacyclononane-1-glutaric acid-4,7-diacetic acid (NODAGA), ethylene diamine tetra-acetic acid (EDTA), diethylene triaminepentaacetic acid (DTPA), cyclohexyl-1,2-diaminetetraacetic acid (CDTA), ethyleneglycol-0,0′-bis(2-aminoethyl)-N,N,N′,N′-tetraacetic acid (EGTA), N,N-bis(hydroxybenzyl)-ethylenediamine-N,N′-diacetic acid (HBED), triethylene tetramine hexaacetic acid (TTNA), hydroxyethyidiamine tnacetic acid (HEDTA), 1,4,8,11-tetraazacyclotetradecane-N,N′,N″,N′″-tetraacetic acid (TETA), and 1,4,7,10-tetraaza-1,4,7,10-tetra-(2-carbamoyl methyl)-cyclododecane (TCMC) and 1,4,7,10-tetraazacyclododececane,1-(glutaric acid)-4,7,10-triacetic acid (DOTAGA).

In preferred embodiments, said chelating agent is selected among the following:

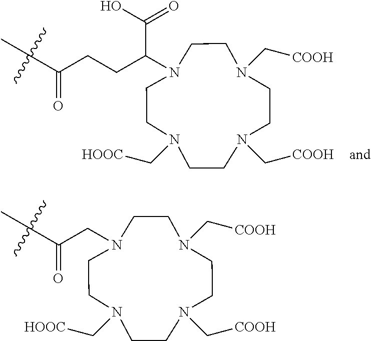 [SMILES: *C(=O)CCC(C(=O)O)N1CCN(CC(=O)O)CCN(CC(=O)O)CCN(CC(=O)O)CC1 ; *C(=O)CN1CCN(CC(=O)O)CCN(CC(=O)O)CCN(CC(=O)O)CC1]

wherein the wavy bond indicates the bond connecting the chelating agent to a linking group of a biocompatible coating forming the nanoparticle.

In a specific embodiment, that may be preferably combined with the previous embodiment, said chelates of rare earth element are chelates of gadolinium and/or bismuth, preferably DOTA or DOTAGA chelating Gd3+ and/or Bi3+. In a more specific embodiment, said chelates of rare earth element are chelates of gadolinium and bismuth with a ratio (moles of gadolinium)/(moles of bismuth) equal to 1.

In specific and preferred embodiments, the ratio of high-Z element per nanoparticle, for example the ratio of rare earth elements, e.g. gadolinium (optionally as chelated with DOTAGA) per nanoparticle, is between 3 and 100, preferably between 5 and 50, for example between 5 and 20, typically around 10.

With such ratio, the nanoparticles have excellent relaxivity and contrast enhancement properties for MR imaging, even when used with MR-Linac with low magnetic field strength, such as 0.35 T or 0.5 T MR-Linac.

In specific embodiments, the hybrid nanoparticles are of core-shell type. Nanoparticles of core-shell type, based on a core consisting of a rare earth oxide and of an optionally functionalized polyorganosiloxane matrix are known (see in particular WO 2005/088314, WO 2009/053644).

The nanoparticles may further be functionalized with molecules which allow targeting of the nanoparticles to specific tissues. Said agents can be coupled to the nanoparticle by covalent couplings, or trapped by non-covalent bonding, for example by encapsulation or hydrophilic/hydrophobic interaction or using a chelating agent.

In one specific embodiment, use is made of hybrid nanoparticles comprising:
a polyorganosiloxane (POS) matrix including, rare earth cations Mⁿ⁺, n being an integer between 2 and 4, optionally partly in the form of a metal oxide and/or oxyhydroxide, optionally associated with doping cations D^{m+}, m being an integer between 2 and 6, D preferably being a rare earth metal other than M, an actinide and/or a transition element;
a chelate covalently bound to the POS via a covalent bond —Si—C—,
the Mⁿ⁺ cations and, where appropriate, D^{m+} cations being complexed by the chelates.

In the case of a structure of core-shell type, the POS matrix forms the superficial layer surrounding the metal cation-based core. Its thickness can range from 0.5 to 10 nm, and can represent from 25% to 75% of the total volume.

The POS matrix acts as protection for the core with respect to the external medium (in particular protection against hydrolysis) and it optimizes the properties of the contrast agents (luminescence, for example). It also allows the functionalization of the nanoparticle, via the grafting of chelating agents and of targeting molecules.

Ultrafine Nanoparticles for Use in the Methods of Treatment of the Disclosure

In a particularly preferred embodiment, said nanoparticles are gadolinium-chelated polysiloxane nanoparticles of the following formula

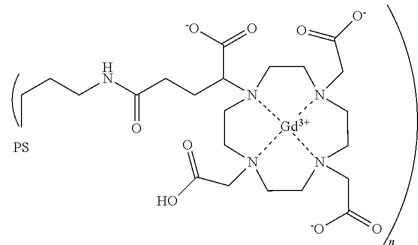

wherein PS is a matrix of polysiloxane, and wherein n is comprised between 5 and 50, typically 5 and 20, and wherein the hydrodynamic diameter is comprised between 1 and 10 nm, for example between 2 and 8 nm, typically about 5 nm.

More specifically, said gadolinium-chelated polysiloxane nanoparticle as described in the above formula is an AGuIX ultrafine nanoparticles as described in the next section.

Such ultrafine nanoparticles that can be used according to the methods of the disclosure may be obtained or obtainable by a top-down synthesis route comprising the steps of:
a. obtaining a metal (M) oxide core, wherein M is a high-Z element as described previously, preferably gadolinium,
b. adding a polysiloxane shell around the M oxide core, for example via a sol gel process,
c. grafting a chelating agent to the POS shell, so that the chelating agent is bound to said POS shell by an —Si—C— covalent bond, thereby obtaining a core-shell precursor nanoparticle, and,
d. purifying and transferring the core-shell precursor nanoparticle in an aqueous solution for dissolution of the metal oxide core,
   wherein the grafted agent is in sufficient amount to complex the cationic form of (M), and wherein the mean hydrodynamic diameter of the resulting ultrafine nanoparticle after dissolution of the core is less than 10 nm, for example, between 1 and 10 nm, typically less than 8 nm, for example between 2 and 8 nm.

In preferred embodiments with complete dissolution of the metal oxide core, these nanoparticles obtained according to the method described above do not comprise a core of metal oxide encapsulated by at least one coating. More details regarding the synthesis of these nanoparticles are given hereafter.

This top-down synthesis method results in observed sizes typically of between 1 and 8 nm, more specifically between 2 and 8 nm. The term then used herein is ultrafine nanoparticles.

Alternatively, another “one-pot” synthesis method is described hereafter to prepare said ultrafine nanoparticles with a mean diameter less than 10 nm, for example, between 1 and 8 nm, typically between 2 and 6 nm.

Further details regarding these ultrafine or core-free nanoparticles, the processes for synthesizing them and their uses are described in patent application WO2011/135101, WO2018/224684 or WO2019/008040, which is incorporated by way of reference.

Process for obtaining preferred embodiments of nanoparticles for use in the methods of treatment of to the disclosure

Generally, those skilled in the art will be able to easily produce nanoparticles used according to the disclosure. More specifically, the following elements will be noted:

For nanoparticles of core-shell type, based on a core of lanthanide oxide or oxyhydroxide, use may be made of a production process using an alcohol as solvent, as described for example in P. Perriat et al., J. Coll. Int. Sci, 2004, 273, 191; O. Tillement et al., J. Am. Chem. Soc., 2007, 129, 5076 and P. Perriat et al., J. Phys. Chem. C, 2009, 113, 4038.

For the POS matrix, several techniques can be used, derived from those initiated by Stoeber (Stoeber, W; J. Colloid Interf Sci 1968, 26, 62). Use may also be made of the process used for coating as described in Louis et al. (Louis et al., 2005, Chemistry of Materials, 17, 1673-1682) or international application WO 2005/088314.

In practice, synthesis of ultrafine nanoparticles is for example described in Mignot et al. Chem. Eur. J. 2013, 19, 6122-6136: Typically, a precursor nanoparticle of core/shell type is formed with a lanthanide oxide core (via the modified polyol route) and a polysiloxane shell (via sol/gel); this object has, for example, a hydrodynamic diameter of around 5-10 nm. A lanthanide oxide core of very small size (adjustable less than 10 nm) can thus be produced in an alcohol by means of one of the processes described in the following publications: P. Perriat et al., J. Coll. Int. Sci, 2004, 273, 191; 0. Tillement et al., J. Am. Chem. Soc., 2007, 129, 5076 and P. Perriat et al., J. Phys. Chem. C, 2009, 113, 4038.

These cores can be coated with a layer of polysiloxane according to, for example, a protocol described in the following publications: C. Louis et al., Chem. Mat., 2005, 17, 1673 and O. Tillement et al., J. Am. Chem. Soc., 2007, 129, 5076.

Chelating agents specific for the intended metal cations (for example DOTAGA for Gd³⁺) are grafted to the surface of the polysiloxane; it is also possible to insert a part thereof inside the layer, but the control of the formation of the polysiloxane is complex and simple external grafting gives, at these very small sizes, a sufficient proportion of grafting.

The nanoparticles may be separated from the synthesis residues by means of a method of dialysis or of tangential filtration, for example on a membrane comprising pores of appropriate size.

The core is destroyed by dissolution (for example by modifying the pH or by introducing complexing molecules into the solution). This destruction of the core then allows a scattering of the polysiloxane layer (according to a mechanism of slow corrosion or collapse), which makes it possible to finally obtain a polysiloxane object with a complex morphology, the characteristic dimensions of which are of the order of magnitude of the thickness of the polysiloxane layer, i.e. much smaller than the objects produced up until now.

Removing the core thus makes it possible to decrease from a particle size of approximately 5-nanometers in diameter to a size below 8 nm, for example between 2-8 nm. Furthermore, this operation makes it possible to increase the number of M (e.g. gadolinium) per nm³ in comparison with a theoretical polysiloxane nanoparticle of the same size but comprising M (e.g. gadolinium) only at the surface. The number of M for a nanoparticle size can be evaluated by virtue of the M/Si atomic ratio measured by EDX. Typically, this number of M per ultrafine nanoparticle may be comprised between 5 and 50.

In one specific embodiment, the nanoparticle according to the disclosure comprises a chelating agent which has an acid function, for example DOTA or DOTAGA. The acid function of the nanoparticle is activated for example using EDC/NHS (1-ethyl-3-(3-dimethylaminopropyl)carbodiimide/N-hydrosuccinimide) in the presence of an appropriate amount of targeting molecules. The nanoparticles thus grafted are then purified, for example by tangential filtration.

Alternatively, the nanoparticles according to the present disclosure may be obtained or obtainable by a synthesis method (“one-pot synthesis method”) comprising the mixing of at least one hydroxysilane or alkoxysilane which is negatively charged at physiological pH and at least one chelating agent chosen from polyamino polycarboxylic acids with
at least one hydroxysilane or alkoxysilane which is neutral at physiological pH, and/or
at least one hydroxysilane or alkoxysilane which is positively charged at physiological pH and comprises an amino function,
   wherein:
the molar ratio A of neutral silane(s) to negatively charged silane(s) is defined as follows: 0≤A≤6, preferably 0.5≤A≤2;
the molar ratio B of positively charged silane(s) to negatively charged silane(s) is defined as follows: 0≤B≤5, preferably 0.25≤B≤3;
the molar ratio C of neutral and positively charged silanes to negatively charged silane(s) is defined as follows 0<C≤8, preferably 1≤C≤4.

According to a more specific embodiment of such one pot synthesis method, the method comprises the mixing of at least one alkoxysilane which is negatively charged at physiological pH, said alkoxysilane being chosen among APTES-DOTAGA, TANED, CEST and mixtures thereof, with
at least alkoxysilane which is neutral at physiological pH, said alkoxysilane being chosen among TMOS, TEOS and mixtures thereof, and/or
APTES which is positively charged at physiological pH,
   wherein:
the molar ratio A of neutral silane(s) to negatively charged silane(s) is defined as follows: 0≤A≤6, preferably 0.5≤A≤2;
the molar ratio B of positively charged silane(s) to negatively charged silane(s) is defined as follows: 0≤B≤5, preferably 0.25≤B≤3;
the molar ratio C of neutral and positively charged silanes to negatively charged silane(s) is defined as follows 0<C≤8, preferably 1≤C≤4.

According to a specific embodiment, the one pot synthesis method comprises the mixing of APTES-DOTAGA which is negatively charged at physiological pH with
at least one alkoxysilane which is neutral at physiological pH, said alkoxysilane being chosen among TMOS, TEOS and mixtures thereof, and/or
APTES which is positively charged at physiological pH,
   wherein:
the molar ratio A of neutral silane(s) to negatively charged silane(s) is defined as follows: 0≤A≤6, preferably 0.5≤A≤2;
the molar ratio B of positively charged silane(s) to negatively charged silane(s) is defined as follows: 0<B≤5, preferably 0.25≤B≤3;
the molar ratio C of neutral and positively charged silanes to negatively charged silane(s) is defined as follows 0<C≤8, preferably 1≤C≤4.

### AGuIX Nanoparticles

In a more particularly preferred embodiment, said gadolinium-chelated polysiloxane based nanoparticle is the ultrafine AGuIX nanoparticle of the formula below

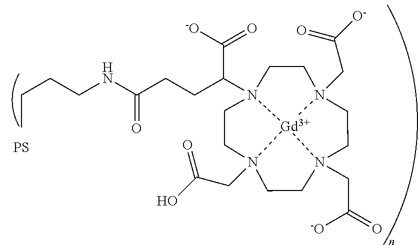

wherein PS is polysiloxane and n is, on average, about 10, and having a hydrodynamic diameter of 5±2 nm and a mass of about 10±1 kDa.

Said AGuIX nanoparticle can also be described by the average chemical formula:

(GdSi₃₋₈C₂₄₋₃₄N₅₋₈O₁₅₋₃₀H₄₀₋₆₀,1-10H₂O)ₙ

Pharmaceutical Formulations of the Nanoparticles for Use According to the Disclosed Methods

When employed as pharmaceuticals, the compositions comprising said high-Z nanoparticles for use as provided herein can be administered in the form of pharmaceutical formulation of a suspension of nanoparticles. These formulations can be prepared as described herein or elsewhere, and can be administered by a variety of routes, depending upon whether local or systemic treatment is desired and upon the area to be treated.

In particular, said pharmaceutical formulations for use as described herein, contain, as the active ingredient, a suspension of high-Z containing nanoparticles, as provided herein, in combination with one or more pharmaceutically acceptable carriers (excipients). In making a pharmaceutical formulation provided herein, the nanoparticle composition may be, for example, mixed with an excipient or diluted by an excipient. When the excipient serves as a diluent, it can be a solid, semi-solid, or liquid material, which acts as a vehicle, carrier, or medium for the nanoparticle composition.

Thus, the pharmaceutical formulations can be in the form of powders, lozenges, elixirs, suspensions, emulsions, solutions, syrups, aerosols (as a solid or in a liquid medium), sterile injectable solutions, sterile packaged powders, and the like.

In specific embodiments, said pharmaceutical formulation for use as described herein, is sterile lyophilized powder, contained in a pre-filled vial to be reconstituted, for example in an aqueous solution for intravenous injection. In specific embodiments, said lyophilized powder comprises, as the active ingredient, an efficient amount of said high-Z containing nanoparticles, typically gadolinium-chelated polysiloxane based nanoparticles, and more specifically AGuIX nanoparticles as described herein. In certain specific embodiments, said lyophilized powder contains either about between 200 mg and 15 g per vial, for example between 280 and 320 mg of AGuIX per vial, typically 300 mg of AGuIX per vial or about between 800 mg and 1200 mg, for example 1 g of AGuIX per vial.

Such powder may further contain one or more additional excipients, and in particular CaCl₂), for example between 0.5 and 0.80 mg of CaCl₂), typically 0.66 mg of CaCl₂).

Said lyophilized powder may be reconstituted in an aqueous solution, typically water for injection. Accordingly, in specific embodiments, said pharmaceutical solution for use according to the present disclosure is a solution for injection, comprising, as the active ingredient, an efficient amount of said high-Z containing nanoparticles, typically gadolinium-chelated polysiloxane based nanoparticles, and more specifically AGuIX nanoparticles as described herein.

For example, said solution for injection for use in the methods as disclosed herein is a solution of gadolinium-chelated polysiloxane based nanoparticles, typically AGuIX nanoparticles, between 50 and 150 mg/mL, for example 80 and 120 mg/mL, typically 100 mg/mL, optionally comprising one or more additional pharmaceutically acceptable excipient, for example between 0.1 and 0.3 mg/mL of CaCl₂), typically 0.22 mg/mL of CaCl₂).

Methods of Treatment of the Present Disclosure

The present disclosure relates to a method of treating a tumor in a subject in need thereof, the method comprising
(i) administering an efficient amount of high-Z element containing nanoparticles having contrast enhancement for magnetic resonance imaging (MRI) and/or radiosensitizing properties, in a subject in need thereof, and,
(ii) exposing said subject to magnetic resonance image-guided radiation therapy by means of a MR-Linac,
   wherein said high-Z element containing nanoparticles are nanoparticles containing an element with an atomic Z number higher than 40, preferably higher than 50, and said nanoparticles have a mean hydrodynamic diameter below 20 nm, for example between 1 and 10 nm, preferably below 8 nm, more preferably between 2 and 8 nm.

The disclosure also relates to high-Z element containing nanoparticles for use in a method of treating a tumor in a subject in need thereof, the method comprising
(i) administering an efficient amount of high-Z element containing nanoparticles having contrast enhancement for magnetic resonance imaging (MRI) and/or radiosensitizing properties, in a subject in need thereof, and,
(ii) exposing said subject to magnetic resonance image-guided radiation therapy by means of MR-Linac,
   wherein said high-Z element containing nanoparticles are nanoparticles containing an element with an atomic Z number higher than 40, preferably higher than 50, and said nanoparticles have a mean hydrodynamic diameter below 20 nm, for example between 1 and 10 nm, preferably below 8 nm, more preferably between 2 and 8 nm.

The disclosure further relates to high-Z element containing nanoparticles for use in the manufacture of a medicament for the treatment of a tumor in a subject in need thereof, the treatment comprising
(i) administering an efficient amount of high-Z element containing nanoparticles having contrast enhancement for magnetic resonance imaging (MRI) and/or radiosensitizing properties, in a subject in need thereof, and,
(ii) exposing said subject to magnetic resonance image-guided radiation therapy by means of a MR-Linac,
   wherein said high-Z element containing nanoparticles are nanoparticles containing an element with an atomic Z number higher than 40, preferably higher than 50, and said nanoparticles have a mean hydrodynamic diameter below 20 nm, for example between 1 and 10 nm, preferably below 8 nm, more preferably between 2 and 8 nm.

As used herein, the term “high-Z element containing nanoparticles” refers to the nanoparticles described in the previous sections.

In preferred embodiments, said high-Z element containing nanoparticles have both contrast enhancement for magnetic resonance imaging (MRI) and radiosensitizing properties. In this regard, preferred embodiments for use in the method of treatment of the present disclosure are the ultrafine nanoparticles, and more preferred embodiments are the AGuIX nanoparticles, as described in the previous sections, which are passively targeted to the tumors and show particularly good contrast enhancement for MR imaging, remarkable radiosensitizing properties.

As used herein, the term “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); and (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease or reducing or alleviating one or more symptoms of the disease. In particular, with reference to the treatment of a tumor, the term “treatment” may refer to the inhibition of the growth of the tumor, or the reduction of the size of the tumor.

The term “patient” and “subject” which are used herein interchangeably refer to any member of the animal kingdom, including mammals and invertebrates. For example, mice, rats, other rodents, rabbits, dogs, cats, swine, cattle, sheep, horses, primates, fish, and humans. Preferably, the subject is a mammal, or a human being, including for example a subject that has a tumor.

In specific embodiments, said tumor is a solid tumor.

Obviously, the present method using MR image-guided radiation therapy together with the nanoparticles as described in the previous section enables a better visualisation and tracking of the lesions, allowing to maximize the volume of the tumor treated by the radiation therapy while minimizing the deleterious effect on the healthy tissue.

The method is particularly well suited for treating tumors in sites affected by inter- and intrafraction motion such as the thorax, abdomen and pelvis.

Hence, in a preferred embodiment, said tumor is localized in one or more of the following sites:
Abdomen, in particular oligometastases, pancreas/duodenum, hepatobiliary, gastric, sarcoma, or other site of the abdomen
Pelvis and lower extremity, in particular, lower gastrointestinal, prostate, bladder, oligometastases, extremity,
Head and neck and brain, and central nervous system,
Thorax, in particular, lung and mediastinum, oesophagus, oligometastases, bone, breast.

In a specific embodiment, said solid tumor is selected from the group consisting of
(i) primary tumor of uterine cervix, rectum, lung, breast, head and neck, prostate, bladder, colorectal, liver, and pancreas cancers or
(ii) bone or hepatic metastases, typically bone metastases undergoing intrafraction movements, such as sternal bones.

The method of the present disclosure for treating cancer comprises a step of administering an efficient amount of said high-Z containing nanoparticles as described above, to the tumor of the subject. The amount administered should be sufficient for the use of the nanoparticles as either MR imaging contrast agent and/or radiosensitizing agent during the MR image-guided radiation therapy. Preferably, the nanoparticles are administered at a sufficient amount for the combined use as MR imaging contrast agent and radiosensitizing agent during the MR image-guided radiation therapy.

The nanoparticles can be administered to the subject using different possible routes such as local (intra-tumoral (IT), intra-arterial (IA)), subcutaneous, intravenous (IV), intradermic, airways (inhalation), intra-peritoneal, intramuscular, intra-thecal, intraocular or oral route.

In specific embodiments, the nanoparticles are administered intravenously. Indeed, the high-Z containing nanoparticles as disclosed herein are advantageously targeted to the human tumors, by passive targeting, for example by enhanced permeability and retention effect.

The nanoparticles may be administered, e.g., 5 minutes, 15 minutes, 30 minutes, 45 minutes, 1 hour, 2 hours, 4 hours, 6 hours, 12 hours, or 24 hours, prior to the administration of the first session of radiation therapy, to the subject with the tumor to be treated.

In a particular embodiment, the method may be preceded by one or more sessions of radiation therapies without the use of any radiosensitizing agent. This may be used for example in prostate cancer, wherein one or more radiation therapy targeting the prostate is carried out prior to administering the nanoparticles as the radiosensitizing agent and delivering radiation more specifically to the tumors by magnetic resonance image-guide radiation therapy.

In another particular embodiment, the method comprises a first tumor pre-filling step of the tumor.

Such pre-filling step comprises administering an effective amount of high-Z element containing nanoparticles as radiosensitizing agent in said subject in need thereof within a period between 2 and 10 days, preferably 2 and 7 days, prior to the first exposure to radiation therapy.

Indeed, considering the remanence of the nanoparticles of the tumor, it may be advantageous to “pre-fill” the tumor with the high-Z element containing nanoparticles with the period between 2 and 10 days, and then administering again the nanoparticles e.g., 5 minutes, 15 minutes, 30 minutes, 45 minutes, 1 hour, 2 hours, 4 hours, 6 hours, 12 hours, 24 hours), prior to the administration of the first irradiation of radiotherapy, to the subject with the tumor to be treated.

Hence, in a particular embodiment, the present method comprises
(i) injecting a first effective amount of high-Z element containing nanoparticles as radiosensitizing agents in said subject in need thereof within a period between 2 and 10 days, preferably between 2 and 7 days, prior to the first irradiation of the tumor,
(ii) injecting a second effective amount of the same or different high-Z element containing nanoparticles within a period between 1 hour to 12 hours prior to the first irradiation of the tumor, and,
(iii) exposing the subject to one or more sessions of magnetic resonance image-guided radiation therapy.

In other embodiments, which may be combined with the previous embodiments, further injections or administrations of nanoparticles can be performed, when appropriate, after one or more sessions of radiation therapy.

Typically, when using fractionated radiation therapy, the nanoparticles may be further injected once every week during multiple sessions of radiation therapy. For example, in a specific embodiment, the method as disclosed herein further comprises at least one additional step of injecting a therapeutically effective amount of the same or different high-Z element containing nanoparticles within 5-10 days after one or more sessions of fractionated MR image-guided radiation therapy, for example 7 days after said injecting step for the first sessions of fractionated MR image-guided radiation therapy.

In preferred embodiments, said nanoparticles are gadolinium-chelated polysiloxane based nanoparticles (e.g. AGuIX nanoparticles) and a therapeutically effective amount administered intravenously at each injecting step is comprised between 50 mg/kg and 150 mg/kg, typically, between 80 and 120 mg/kg, for example 100 mg/kg.

The Step of Image-Guided Radiation Therapy

According to the method of the present disclosure, the subject is exposed to magnetic resonance image-guided radiation therapy by Magnetic Resonance Imaging Guided Linear Accelerator (MR-Linac).

As used herein, the term “radiation therapy”, also referred as “radiotherapy” is used for the treatment of diseases of oncological nature with irradiation corresponding to ionizing radiation. Ionizing radiation deposits energy that injures or destroys cells in the area being treated (the target tissue) by damaging their genetic material, making it impossible for these cells to continue to grow. Typically, said ionizing radiations are photons, e.g; X-rays. Depending on the amount of energy they possess, the rays can be used to destroy cancer cells on the surface of or deeper in the body. The higher the energy of the X-ray beam, the deeper the X-rays can go into the target tissue.

Linear accelerators produce X-rays of increasingly greater energy. The use of machines to focus radiation (such as X-rays) on a cancer site is called external beam radiotherapy.

Ionizing radiations are typically of 2 MeV to 25 MeV in particular between 4 MeV and 18 MeV, typically 4 MeV or 6 MeV with MR-Linac.

As used herein, the term “magnetic resonance image-guided radiation therapy” refers to the combined use of a magnetic resonance imaging unit with a radiation therapy unit, allowing real-time imaging of target volumes and organs at risk before and during treatment delivery with replanning as necessary. Magnetic resonance image-guided radiation therapy is particularly useful in sites affected by inter- and intrafraction motion such as the thorax, abdomen and pelvis. Typically, organ at risk and target visualization can be improved with magnetic resonance image-guided radiation therapy compared to cone beam computerized tomography, which can permit plan adaptation and reduction of toxicity. This technique may use automated beam gating for precise and accurate dosing. When the tumor moves, the beam automatically stops. Clinicians can therefore shrink margins with confidence while escalating dose.

Any MR-Linac for image-guided radiation therapy may be used in the methods of treatment of the present disclosure.

The MR-Linac as currently used includes perpendicular beam-field systems (e.g. Elekta and Viewray) which are now commercial products. Other systems include inline orientation (Aurora-RT) and both perpendicular and inline orientation (Australian). The field strength of the current MR-Linac systems vary from 0.35 T; for example the MRIdian (Viewray), 0.5T (Aurora-RT, MagneTx), and 1.5T (Unity, Elekta). Further details of the MR-Linac systems and their mode of use are described in Liney et al Clinical Oncology 30 (2018) 686-691.

In preferred embodiment, a MR-Linac used in the method of the present disclosure has a magnetic field strength of 0.5 T or lower, for example 0.35 T. Such embodiment is particularly preferred with ultrafine nanoparticles or AGuIX nanoparticles as described in the previous sections.

Typically, a session of magnetic resonance image-guided radiation therapy with MR-Linac includes the following step:

1. A Simulation Step

Typically, prior to the MR image-guided radiation therapy, a pre-treatment computerized tomography (CT) and MRI is acquired. The target and organs at risk may be contoured by a radiation oncologist on the pre-treatment data.

2. A Repositioning Step, if Necessary an Adaptative Planning Step

At the beginning of every treatment session the patient is positioned on the treatment table. a fresh MRI scan is taken, and compared with the original scan used to create the radiation treatment plan. If anything on the scan has changed, the radiation treatment plan may be adapted to account for movement of the tumor and organs.

3. A Treatment Delivery Step

Once the highly specialized team is satisfied with the radiation treatment plan and targeting, the patient will receive his treatment.

The radiation delivery on the MR-LINAC is fully integrated with the MRI. This technology means the system can deliver treatment radiation beams and monitor the target area at the same time. The radiation beams are precisely shaped to maximize the dose to the target while minimizing the dose to the surrounding healthy tissue.

While the radiation beam is on, the MR-LINAC is capturing a constant video of the tumor and/or nearby organs with its MRI and acting on them at sub-second speed. If the tumor or a critical organ moves beyond a boundary defined by the physician, the radiation beam automatically pauses; when the target moves back into the predefined boundary, treatment automatically resumes. Hence, the correct amount of radiation is delivered to the correct location.

Examples of protocols for use of MR-Linac in magnetic resonance image-guided radiation therapy for treating tumors are disclosed in Fischer-Valuck et al, 2017 (Advances in Radiation Oncology, 2, 485-493), Henke L E, et al., Magnetic Resonance Image-Guided Radiotherapy (MRIgRT): A 4.5-Year Clinical Experience, Clinical Oncology (2018), https://doi.org/10.1016/j.clon.2018.08.010;

According to the current practice, standard MRI contrast agents are administered prior to the magnetic resonance image-guided radiation therapy at each session to improve contrast enhancement. Examples of such standard contrast agents that have been used with MR-Linac include cyclic agents for example, gadobenic acid (MultiHance), gadoteric acid (Dotarem), and gadobutrol (Gadovist).

In the method of the present disclosure, the high-Z containing nanoparticles administered to the subject prior to image-guided radiation therapy is used either as contrast agent for MR imaging or as radiosensitizing agent for radiation therapy, or preferably, both as contrast agent for MR imaging and as radiosensitizing agent.

Contrary to the standard contrast agent used in the prior art, the inventors indeed noticed that the high-Z containing nanoparticles as disclosed herein (and more specifically. AGuIX nanoparticles or other Gd-based ultrafine nanoparticles) have the following advantages:
They can be used both as contrast agent for MR imaging and as radiosensitizing agent, allowing a single injection step prior to the radiation therapy.
They have a particularly long remanence in the tumor of several days, allowing to avoid administering such nanoparticles at each session.
They have a high relaxivity, likely due to the high number of Gd per particle (usually between 5 and 50 as compared to 1 for other conventional contrast agent such as Dotarem), which allow for high quality MR imaging, and make them particularly suitable for use with MR-Linac, preferably of low magnetic field strength, for example of 0.5T or lower, and typically 0.35T.

In a preferred embodiment, considering the observed remanence of the high-Z containing nanoparticles in the tumor after a single intravenous injection, the subject may be exposed to multiple sessions of magnetic resonance image-guided radiation therapy, without further administration of a contrast agent for MRI. Typically, said subject is exposed to at least 2 sessions of magnetic resonance image-guided radiation therapy, without further administration of a contrast agent for MRI.

In a particular embodiment, said subject is exposed to 2, 3, 4, 5, 6, or 7 sessions of magnetic resonance image-guided radiation therapy after a single administration of an efficient amount of said high-Z containing nanoparticles. For example, said subject may be exposed to 2 or more sessions of magnetic resonance image-guided radiation therapy within 5-7 days. In certain embodiments, a minimum timeline of 2 or 3 days may be observed between each session.

A person of ordinary skill in the art of MR image-guided radiation therapy knows how to determine an appropriate dosing and application schedule, depending on the nature of the disease and the constitution of the patient. In particular, the person knows how to assess dose-limiting toxicity (DLT) and how to determine the maximum tolerated dose (MTD) accordingly.

The amount of radiation used in photon radiation therapy is measured in gray (Gy), and varies depending on the type and stage of cancer being treated. For curative cases, the typical total dose for a solid tumor ranges from 20 to 120 Gy, typically 25 to 100 Gy.

Many other factors are considered by radiation oncologists when selecting a dose, including whether the patient is receiving chemotherapy, patient co-morbidities, whether radiation therapy is being administered before or after surgery, and the degree of success of surgery.

The total dose is typically fractionated (spread out over time). Amount and schedules (planning and delivery of ionizing radiations, fraction dose, fraction delivery schema, total dose alone or in combination with other anti-cancer agents etc) is defined for any disease/anatomical site/disease stage patient setting/age and constitutes the standard of care for any specific situation.

A typical conventional fractionation schedule for adults for the methods of the present disclosure may be 1.8 to 3.0 Gy per day, five days a week, for example for 2 to 8 consecutive weeks. In specific embodiments, said radiotherapy consists of exposing the subject to a total dose of ionizing radiations between 25 and 80 Gy, for example 30 Gy.

Considering the combined effect of nanoparticles and ionizing radiations according to the present method obtained with high dose of ionizing radiations, in one specific embodiment, the dose of ionizing radiations exposed to the tumor of the patient is advantageously hypofractionated. For example, a dose per fraction of at least 3 Gy, and for example between about 3 Gy to about 20 Gy, or between 5 and 7 Gy is exposed to the tumor of the patient and radiation total dose is delivered in few fractions (typically, but not necessarily no more than 10 fractions, for example between 1 and 10 fractions).

In specific embodiment where the subject is suffering from pancreas cancer, the radiation therapy applied of the herein disclosed methods comprises exposing the subject to 6 sessions of MR-image guided radiation therapy with MR-Linac system, with a fraction of 8 Gy per session.

In other specific embodiments where the subject is suffering from prostate cancer, the radiation therapy applied of the herein disclosed methods comprises exposing the subject to 5 sessions of MR-image guided radiation therapy with MR-Linac system, with a fraction of 9 Gy per session applied to the prostate without radiosensitizing agent, and then to a boost of 4 additional sessions for treating the prostate tumor only using MR-image guided radiation therapy with MR-Linac system, typically with a fraction of 10 Gy per session. The high-Z containing nanoparticles (for example ultrafine or AGuIX nanoparticles) is administered only for the 4 last sessions.

In other specific embodiment where the subject is suffering from hepatic metastases, the radiation therapy applied of the herein disclosed methods comprises exposing the subject to 6 sessions of MR-image guided radiation therapy with MR-Linac system, with a fraction of 9 Gy per session.

In other specific embodiment where the subject is suffering from lymph node metastases, the radiation therapy applied of the herein disclosed methods comprises exposing the subject to 5 sessions of MR-image guided radiation therapy with MR-Linac system, with a fraction of 6 Gy per session.

In other specific embodiment where the subject is suffering from bone metastases, the radiation therapy applied of the herein disclosed methods comprises exposing the subject to 6 sessions of MR-image guided radiation therapy with MR-Linac system, with a fraction of 9 Gy per session.

Typically for the above embodiments, ultrafine gadolinium-based nanoparticles, and more preferably Aguix nanoparticles, as described in the previous sections, are used as the MR imaging contrast agent and radiosensitizing agent for one or more MR-guided radiation therapy sessions.

Further non-limiting details are provided in the Examples.

Combination Therapy with the Method of the Present Disclosure

The nanoparticles for use as disclosed herein may be administered as the sole active ingredient or in conjunction with, e.g. as an adjuvant to or in combination to, other drugs e.g cytotoxic, anti-proliferative, or other anti-tumor agents, e.g. for the treatment or prevention of cancer disorders, as mentioned above.

Suitable cytotoxic, anti-proliferative or anti-tumor agents may include without limitation cisplatin, doxorubicin, taxol, etoposide, irinotecan, topotecan, paclitaxel, docetaxel, epothilones, tamoxifen, 5-fluorouracil, methotrexate, temozolomide, cyclophosphamide, tipifarnib, gefitinib, erlotinib, imatinib, gemcitabine, uracil mustard, chlormethine, ifosfamide, melphalan, chlorambucil, pipobroman, triethylenemelamine, busulfan, carmustine, lomustine, streptozocin, dacarbazine, floxuridine, cytarabine, 6-mercaptopurine, 6-thioguanine, fludarabine phosphate, oxaliplatin, folinic acid, pentostatin, vinblastine, vincristine, vindesine, bleomycin, dactinomycin, daunorubicin, epirubicin, idarubicin, mithramycin, deoxycoformycin, mitomycin-C, L-asparaginase, teniposide.

In some embodiments, the additional therapeutic agent is administered simultaneously with a composition provided herein. In some embodiments, the additional therapeutic agent is administered after administration of the composition provided herein. In some embodiments, the additional therapeutic agent is administered prior to administration of the composition herein. In some embodiments, the composition provided herein is administered during a surgical procedure. In some embodiments, the composition provided herein is administered in combination with an additional therapeutic agent during a surgical procedure.

The additional therapeutic agents provided herein can be effective over a wide dosage range and are generally administered in an effective amount. It will be understood, however, that the amount of the therapeutic agent actually administered will usually be determined by a physician, according to the relevant circumstances, including the condition to be treated, the chosen route of administration, the actual compound administered, the age, weight, and response of the individual subject, the severity of the subjects symptoms, and the like.

Other aspects and advantages of the method of the disclosure will become apparent in the following examples, which are given for purposes of illustration only.

### EXAMPLES

### Example 1: First-In-Human Trial of Gd-Based Theranostic Nanoparticles: Uptake and Biodistribution in Patients with 4 Types of Brain Metastasis

1.1 Materials and Methods

Study Design

This study is part of a prospective dose escalation phase I-b clinical trial to evaluate the tolerance of the intravenous administration of radiosensitizing AGuIX nanoparticles in combination with whole brain radiotherapy for the treatment of brain metastases. The Nano-Rad trial (Radiosensitization of Multiple Brain Metastases Using AGuIX Gadolinium Based Nanoparticles) was registered as NCT02820454. Here, we report the findings of the MRI protocol applied to the 15 recruited patients. The objectives assigned to this MRI ancillary study were to i) assess the distribution of AGuIX nanoparticles in brain metastases and surrounding healthy tissues and ii) to measure the T₁-weighted contrast enhancement and nanoparticle concentration in brain metastases and surrounding healthy tissues after intravenous administration of AGuIX nanoparticles (Verry C, et al. BMJ Open. 9:e023591 (2019)).

Patient Selection

Patients with multiple brain metastases ineligible for local treatment by surgery or stereotactic radiation were recruited. Inclusion criteria included: i) minimum age of 18 years, ii) secondary brain metastases from a histologically confirmed solid tumor, iii) no prior brain irradiation, iv) no renal insufficiency (glomerular filtration rate >60 mL/min/1.73 m²), v) normal liver function (bilirubin <30 μmol/L; Alkaline phosphatase <400 UI/L; Aspartate aminotransferase (AST) <75 UI/L; Alanine aminotransferase (ALT) <175 UI/L).

Trial Design

The main steps of the trial protocol were as follows. At D0, patients underwent a first imaging session (see MRI protocol in next paragraph) including the intravenous bolus injection of Dotarem (gadoterate meglumine) at a dose of 0.2 mL/kg (0.1 mmol/kg) body weight. 1 to 21 days after the first imaging session (depending on patient availability and radiation therapy planning), the patients were administered intravenously with solution of AGuIX nanoparticles at doses of 15, 30, 50, 75 or 100 mg/kg body weight. The date of AGuIX nanoparticles administration is referred as D1.

Synthesis of AGuIX Nanoparticles

AGuIX nanoparticles have been obtained by a six step synthesis. The first step is the formation of a gadolinium oxide core by addition of soda on gadolinium trichloride in diethylene glycol. Second step is growth of a polysiloxane shell by adding TEOS and APTES. After maturation, DOTAGA anhydride is added for reaction with free amino functions present at the surface of the inorganic matrix. After transfer to water, dissolution of the gadolinium oxide core is observed and gadolinium is chelated by DOTAGA at the surface of the matrix. Then, fragmentation of the polysiloxane matrix in ultrasmall AGuIX nanoparticles is observed. Last step is freeze drying of the nanoparticles.

The theranostic agent is composed of a polysiloxane network surrounded by gadolinium cyclic ligands, derivatives of DOTA (1,4,7,10-tetraazacyclododecane acid-1,4,7,10-tetraacetic acid), covalently grafted to the polysiloxane matrix. Its hydrodynamic diameter is 4±2 nm, its mass is about 10 kDa and is described by the average chemical formula (GdSi₃₋₈C₂₄₋₃₄N₅₋₈O₁₅₋₃₀H₄₀₋₆₀, 1-10 H₂O)ₙ. On average each nanoparticle presents on its surface 10 DOTA ligands which chelate core gadolinium ions. The longitudinal relaxivity r₁ at 3 Tesla is equal to 8.9 mM⁻¹·s⁻¹ per Gd³⁺ ion, resulting in a total r₁ of around 89 mM⁻¹·s⁻¹ per AGuIX nanoparticle. The same MRI session, without injection of gadoterate meglumine, was performed 2 hours post administration of the nanoparticles. The patients then underwent a whole brain radiation therapy (30 Gy delivered in 10 sessions of 3 Gy). 7 days (D8) and 4 weeks (D28) after the AGuIX nanoparticles were administered, a similar MRI session was performed for each patient.

MRI Protocol

The MRI acquisitions were performed on a 3 Tesla Philips scanner. 32-channel Philips head coil were used. Patients underwent identical imaging protocol including the following MRI sequences: i) 3D weighted gradient echo sequence, ii) 3D FLASH sequence with multiple flip angles, iii) susceptibility-weighted imaging (SWI) sequence, iv) Fluid Attenuated Inversion Recovery (FLAIR) sequence, v) Diffusion-weighted imaging (DWI) sequence. Some of these imaging sequences are recommended when following the RECIST (Response Evaluation Criteria in Solid Tumors) and RANO (Response Assessment in Neuro-Oncology) criteria for assessing brain metastases response after radiotherapy (24, 25). The 3D T₁-weighted imaging sequence provides high-resolution contrast-enhanced images of healthy tissue and brain metastases following MRI contrast agent administration. The 3D FLASH sequence is repeated several times with a different flip angle for computing T₁ relaxation times and contrast agent concentration. The SWI sequence is used for detecting the presence of hemorrhages. The FLAIR sequence is applied for monitoring the presence of inflammation or edema. Finally, the DWI sequence can be applied for detecting abnormal water diffusion in tissue or brain metastases.

The total acquisition time ranged between 30 minutes and 40 minutes depending on patient-adjusted imaging parameters. The key features and the main acquisition parameters of these imaging sequences are detailed in Supplementary Materials section.

Image Processing and Quantification Pipeline

MRI analyses were performed using an in-house computer program called MP3 (https://github.com/nifm-gin/MP3) developed by the GIN Laboratory (Grenoble, France) and running under Matlab® software. Image analyses include counting and measurements of metastases, quantification of contrast enhancement, relaxation times and concentration of nanoparticles. Following RECIST and RANO criteria, solely metastases with longest diameter above 1 cm were considered as measureable and were retained in subsequent analyses. The MRI enhancement, expressed in percentage, was defined as the ratio of the MRI signal amplitude post contrast agent administration over the MRI signal amplitude pre contrast agent administration; the MRI signal amplitude being measured in the 3D T₁-weighted image dataset. The T₁ relaxation times were derived from the 3D FLASH images obtained at four different flip angles. The concentration of nanoparticles in brain metastases was derived from the variations of T₁ relaxation times pre and post contrast agent administration and from the known relaxivity of the nanoparticles.

A 3D image rendering was performed using the BrainVISA/Anatomist software (http://brainvisa.info) developed at NeuroSpin (CEA, Saclay, France). To better visualize the location of the different metastases, the Morphologist pipeline of BrainVISA was used to generate the meshes of both the brain and the head of each patient.

Statistical Analysis

All analyses were performed using GraphPad Prism (GraphPad Software Inc.). Unless specified, significance was fixed at a 5% probability level. Unless specified, all of the data are presented as mean value ±SD.

1.2 Results

Administered AGuIX Gd-Based Nanoparticles Induce MRI Contrast Enhancement in all Four Types of Brain Metastases

The patient recruitment resulted into the inclusion of four types of brain metastases, namely NSCLC (non-small-cell lung carcinoma) N=6, breast N=2, melanoma N=6 and colon cancer N=1. All the patients were successfully injected with the theranostic nanoparticles AGuIX (as described in Materials and Methods) at each escalation step of administered dose (N=3 for 15, 30, 50, 75 and 100 mg/kg body weight).

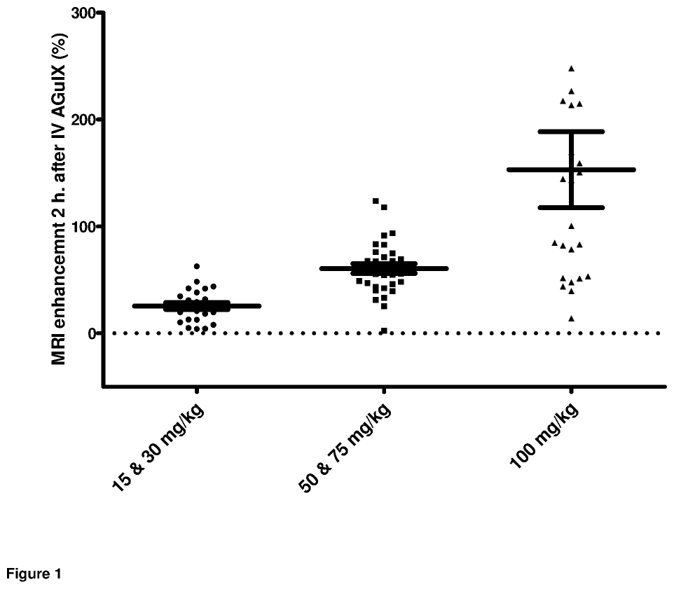

At D1, two hours after AGuIX injection, MRI signal enhancements were observed for all types of brain metastases, all patients and all doses administered. Within the region of interest drawn around each metastasis, MRI signal enhancements were found to increase with the administered dose of AGuIX nanoparticles (FIG. 1). Signal enhancements, averaged over all measurable metastases (longest diameter greater than 1 cm), were equal to 26.3±15.2%, 24.8±16.3%, 56.7±23.8%, 64.4±26.7% and 120.5±68% for AGuIX doses of 15, 30, 50, 75 and 100 mg/kg body weight respectively. The MRI enhancement was found to linearly correlate with the injected dose (slope 1.08, R²=0.90) (data not shown).

Gd-Based Nanoparticles Demonstrate MRI Enhancement of Brain Metastases Equivalent to that of a Clinically-Used Contrast Agent

For each patient, the MRI enhancement was also measured at DO 15 min after injection of a clinically-approved Gd-based contrast agent (Dotarem®, Guerbet, Villepinte, France). Averaged over all measurable metastases with longest diameter larger than 1 cm, the MRI enhancement was equal to 182.9±116.2%. This MRI enhancement, observed 15 min after injection, is in the same order of magnitude than the one observed 2 h after the administration of the highest dose of AGuIX nanoparticles.

The detection sensitivity of AGuIX nanoparticles, defined as their ability of enhancing MRI signal in measurable brain metastases was assessed for all administered doses and compared to the sensitivity of the clinically-used contrast agent Dotarem®. Expressed as a percentage of Dotarem sensitivity, the AGuIX nanoparticles sensitivity was equal to 12.1, 19.5, 34.2, 31.8 and 61.6% for injected doses of 15, 30, 50, 75 and 100 mg/kg body weight respectively.

Concentration of AGuIX Nanoparticles can be Quantified in Brain Metastases

The multi flip-angle 3D FLASH acquisitions were successfully used to compute pixel-wise maps of T₁ values (data not shown) and to enable quantification of the longitudinal relaxation time over regions of interest. The decrease of T₁ relaxation times in brain metastases, induced by the uptake of AGuIX nanoparticles, is clearly shown in these T₁ maps. As expected, the decreases of T₁ values are co-localized with the contrast-enhanced brain metastases.

The concentrations of AGuIX nanoparticles in contrast-enhanced metastases were computed based on changes in T₁ values following their administration. The measurements of AGuIX concentration were performed in metastases with longest diameter larger than 1 cm for the patients administered with a dose of 100 mg/kg body weight. The mean AGuIX concentration in the brain metastases was measured to be 57.5±14.3, 20.3±6.8, 29.5±12.5 mg/L in patient #13, #14 and #15 respectively.

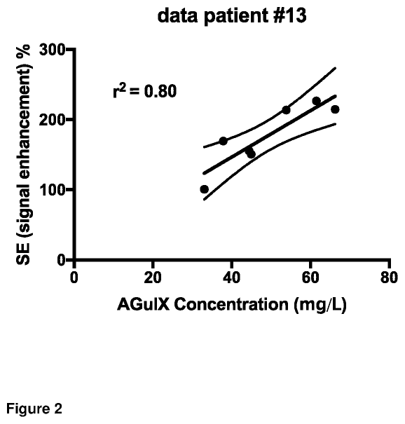

The correlation between MRI enhancement and nanoparticles concentration was assessed for patients with the highest (100 mg/kg) administered dose. The correlation is exemplified in FIG. 2 with MRI data from patient #13 with NSCLC metastases. A strong positive correlation between the two MRI parameters was observed with a relationship close to linearity in the range of measured values.

For each patient, the MRI enhancement and T₁ values were assessed in brain regions of interest free of visible metastases (three representative regions of interest per patient, with a similar size for all patients). No significant MRI enhancement and no T₁ variations were observed in any of these healthy brain regions.

MRI Enhancements are Observed One Week after Nanoparticle Administration

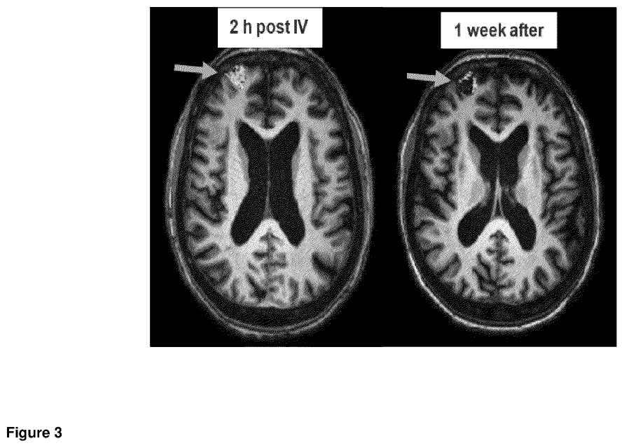

For patients administered with the largest dose (100 mg/kg body weight), persistence of MRI enhancement was noticed in measurable metastases (longest diameter greater than 1 cm) at D8, i.e. up to one week after administration of AGuIX nanoparticles as shown in FIG. 3. The mean MRI enhancements in metastases were measured equal to 32.4±10.8%, 14±5.8% and 26.3±9.7% for patient #13, #14 and #15 respectively. As a point of comparison, the mean MRI enhancements at D1 were equal to 175.8±45.2%, 58.3±18.4% and 154.1±61.9% for patients #13, #14 and #15 respectively. Due to low T₁ variations, the concentration of AGuIX nanoparticles could not be computed. Based on the observed correlation between MRI enhancement and nanoparticles concentration, an upper limit of 10 μM can be estimated for the AGuIX concentration at D8 in brain metastases. No noticeable MRI enhancement was observed in any patient at D28, 4 weeks after the administration of AGuIX nanoparticles.

### Discussion

The occurrence of brain metastases is a common event in the history of cancer and negatively affects the life expectancy of patients. For patients with multiple brain metastases, whole brain radiation therapy (WBRT) remains the standard of care. However, the median overall survival is less than six months and new approaches need to be developed to improve the treatment efficacy for these patients. The use of radiosensitizing agents is thus of great interest. The in vivo theranostic properties (radiosensitization and diagnosis by multimodal imaging) of AGuIX nanoparticles were previously demonstrated in preclinical studies performed on eight tumor models in rodents (F. Lux, et al. Br J Radiol. 18:20180365 (2018)), and particularly in brain tumors (G. Le Duc, et al. ACS Nano. 5, 9566-9574 (2011), C. Verry C, et al. Nanomedicine 11, 2405-2417 (2016)). The clinical evaluation of the diagnostic value of the AGuIX nanoparticles for brain metastases was one of the secondary objectives of the clinical trial Nano-Rad. The target dose for the radiotherapeutic application of the AGuIX nanoparticles in patients is 100 mg/kg and for this reason the conclusions and perspectives of this study focus essentially on this dose.

The largest dose of AGuIX nanoparticles (100 mg/kg body weight or 100 μmol/kg body weight Gd³⁺) administered to the patients corresponds to the amount of chelated gadolinium ions Gd³⁺ injected in one dose of clinically-used MRI contrast agent such as Dotarem® (100 □mol/kg body weight Gd³⁺). It is therefore appropriate to compare the MRI enhancements observed in metastases with the largest AGuIX dose to a dose of Gd-based contrast agent used in clinical routine.

In this study, there was a 2-hours delay between the nanoparticle administration and the MRI acquisitions for monitoring the patient's response to the injection. With a mean nanoparticle plasma half-life of about 1 hour, this delay results in an 86% decrease of the nanoparticles concentration in the plasma. In contrast, there was only a 15-minutes delay between the Dotarem® injection and the MRI acquisition. Despite this significant clearance of nanoparticles and the decrease of concentration in the patient's bloodstream, the MRI enhancement at the highest nanoparticle dose is close to that observed with the clinical contrast agent.

This remarkable diagnostic performance of AGuIX nanoparticles to enhance the MRI signal in brain metastases may be attributed to two independent factors. The first factor is related to the intrinsic magnetic properties of nanoparticles. Their larger diameter and molecular weight, as compared to clinical Gd-based contrast agent, result in a higher longitudinal relaxation coefficient r₁ and thus an increased ability to modify the intensity of the MRI signal. Concretely, the r₁ values for AGuIX nanoparticles and Dotarem® are respectively equal to 8.9 and 3.5 mM⁻¹·s⁻¹ per Gd³⁺ ion at a magnetic field of 3 Tesla (B. R. Smith, S. S. Gambhir. Chem Rev. 117, 901-986 (2017)).

The second factor may be related to the ability of AGuIX nanoparticles to passively accumulate in brain metastases. This passive targeting phenomenon takes advantage of the so-called Enhanced Permeability and Retention (EPR) effect which postulates that the accumulation of nano-objects in tumors is due to both defective and leaky tumor vessels and to the absence of effective lymphatic drainage (A. Bianchi, et al. MAGMA. 27, 303-316 (2014)). The passive targeting of tumors by AGuIX nanoparticles has been consistently observed in previous investigations of animal models of cancer. In a mouse model of multiple brain melanoma metastases, internalization of AGuIX nanoparticles in tumor cells was reported and the presence of nanoparticles in brain metastases was still observed 24 hours after intravenous injection to the animals (Kotb, A. et al. Theranostics 6(3):418-427 (2016)). At the highest 100 mg/kg dose, all metastases with a diameter larger than 1 cm were contrast-enhanced up to 7 days after the nanoparticles were administered.

The Persistence of MRI Signal Enhancement in Metastases One Week after Administration Highlights this Accumulation and Delayed Clearance of Nanoparticles from the Metastasis. To the Best of the Inventor's Knowledge, there is No Report in the Literature of Such Late MRI Enhancement in Metastases after Administration of Clinically Used Gd-Based Contrast Agents.

A dose escalation was included in the design of this first-in-man clinical trial and the patients were thus administered with five increasing dose levels of AGuIX nanoparticles. From the linear correlation observed between the signal enhancement in metastases and the administered nanoparticles concentration, it can be concluded that the dose of nanoparticles—in the range of investigated doses—is not a limiting factor for the passive targeting of metastases. Importantly, despite the limited number of patients participating in this first clinical study, these initial results show that nanoparticles uptake and signal enhancement are present in the four types of investigated metastases (NSCLC, melanoma, breast and colon), regardless of the injected dose of nanoparticles.

Considering the radiosensitizing properties of AGuIX nanoparticles, it is key to evaluate and possibly quantify the local concentration of nanoparticles accumulated in metastases. To that end, the MRI protocol included a T₁ mapping imaging sequence from which the nanoparticles concentration was derived. The concentration values obtained in this clinical study can be put in perspective with those obtained in pre-clinical studies in animal models of tumor. The computed concentration of AGuIX nanoparticles in the NSCLC and breast cancer metastases of the three patients injected with the highest dose varied between 8 and 63 mg/L, corresponding to a concentration range of Gd³⁺ ions between 8 and 63 μM in brain metastases. In the three aforementioned patients, the % ID/g is ranging between 8 and 63%. The same order of magnitude was found for the % ID/g in the two above-mentioned MRI pre-clinical studies, with 28% and 45% ID/g respectively. Interestingly, these concentrations are obtained with a delay post-injection (several hours) that is compatible with the setup of a radiotherapy session.

In this study, we evaluated as well the relationship between the nanoparticles concentration and the MRI signal enhancement SE obtained using a robust T₁-weighted 3D MRI sequence. In the range of measurable nanoparticles concentration in metastases, a linear relationship between the MRI enhancement and the nanoparticles concentration is observed with the acquisition protocol used in this study. Hence, with the specific protocol used in this study, the MRI enhancement can be used as a robust and simple index for assessing the concentration of AGuIX nanoparticles.

While metastasis targeting is beneficial for both diagnosis and radiosensitization purposes, it is desirable to maintain nanoparticles at low concentration in healthy surrounding tissues. In this respect, no MRI enhancement could be observed in the metastasis-free brain tissues two hours after the highest dose of AGuIX nanoparticles was administered. This lack of enhancement is consistent with the rapid clearance of nanoparticles measured in patient's plasma and is a positive indication of the innocuousness of the nanoparticles for the healthy brain.

In summary, the preliminary results of the clinical trial reported here demonstrate that an intravenous injection of Gd-based nanoparticles is effective for enhancing different types of brain metastases in patients.

In addition to this, the preliminary results of the phase 1 clinical trial demonstrate a good tolerance of intravenous injection of AGuIX nanoparticle up to the 100 mg/kg dose selected for this study.

Lastly, the persistence of the nanoparticles in the tumor at day 8 support a protocol allowing a single injection of such nanoparticles as contrast and radiosensitizing agent prior to the first irradiation, for example using image-guided radiation therapy as described in Example 2, and subsequent sessions of radiation therapy within 5-7 days following the single injection of the nanoparticles.

### Example 2: Synopsis for Evaluating the Feasibility and Tolerance of Hypofractionated Radiotherapy Delivered by MR-Linac System Combined with the Use of AGuIX Nanoparticles as a Radiosensitizing and Contrast Agent

2.1 Rationale and Targeted Patient Profile

Magnetic Resonance Imaging Guided Linear Accelerator (MR-Linac) uses magnetic resonance imaging, or MRI, together with radiotherapy to treat cancers throughout the body, with specific advantages for tumours located inside soft-tissue. The system can deliver treatment radiation beams and monitor the target area at the same time. The combination of technologies gives to radiation oncologists a greater control over the delivery of radiation because they can see the internal anatomy and tumour. They can fine-tune the radiation treatment plan and personalize and adapt each treatment. However, according to tumour types or localisation, a contrast agent use is necessary to highlight and track tumour during treatment.

Contrast agents currently used, have to be injected before each RT fraction.

AGuIX NP is a potential theranostic agent with its capacity to increase tumour radiosensibility and to increase tumour contrast for MRI scan. Moreover, AGuIX may increase this tumour contrast during several days allowing a single weekly injection.

2.2 Objective of the Study

The primary objective is to evaluate the use of AGuIX as contrast agent to track tumor by MRI scan of MR-Linac device during 2 or 3 sessions per week, using a single AGuIX injection per week.

Associated Endpoint: Contrast agent efficacy evaluation from MRI scan performed using MR-Linac at different time points after injection (from 1 hour to 5 days).

Secondary Objectives:

To evaluate safety of hypofractionated RT delivered using MR-linac and AGuIX combination. (Endpoint: Acute adverse events according to CTCAE-V5.0 up to three months after the end of radiation therapy)

To evaluate the progression free survival (PFS) following hypofractionated RT delivered using MR-Linac and AGuIX combination. (Endpoint: PFS: the time from randomization to the first occurrence of disease progression, as determine by the investigator according to RECIST V1.1 or death from any cause, whichever occurs first.

2.3 Eligibility Criteria/Subject Characteristics

Inclusion Criteria
25-18 years old
ECOG Status: 0 or 1
Patients with a primary tumor of prostate or pancreas, lymph nodes relapse, hepatic metastases or primary location, bone metastases, typically bone metastases undergoing intrafraction movements, such as sternal bones.
Indication of hypofractionated radiation therapy

Non-Inclusion Criteria
Previous radiation therapy on the same target
Contraindication for MRI Scan
Allergic to contrast agents

2.4 Treatment(s) Used in the Study

Name of drug/treatment and trade name: AGuIX

Chemical name (DCI): Gadolinium-chelated polysiloxane based nanoparticles

Pharmaceutical form: sterile lyophilized off-white powder containing 300 mg AGuIX as active ingredient (300 mg of AGuIX/vial). Each vial contains 0.66 mg of CaCl₂), as an inactive ingredient. The drug product is supplied in a single-use 10 mL clear glass vial with a bromobutyl rubber stopper.

Preparation procedure: reconstitution of the solution with 3 mL of water for injection to obtain a solution of AGuIX at 100 mg/mL. The pH of the solution is then at 7.2±0.2.

One hour after reconstitution with water for injection, the reconstituted solutions will be taken into a syringe, before being injected using a syringe pump.

Administration minimum 1 hour after reconstitution and within 24 hours maximum.

AGuIX solution will be administered in a half-day after reconstitution, however, the nanoparticles must be stored at [+2° C.; +8° C.] and administered within a maximum of 24 hours after reconstitution.

Intravenous administration by slow infusion (2 mL/min) with a syringe pump

Dose per administration: 100 mg/kg, 1 mL/kg

2.5 Treatment and Associated Procedures

Radiotherapy:

Radiotherapy Schemes According to the Location Treated:
Pancreatic cancer: 6 fractions of 8 Gy
Prostate cancer: 5 fractions of 9 Gy in the prostate followed by a boost of 4 fractions of 10 Gy in the tumor (AGuIX use only for the last 4 fractions)
Hepatic metastasis: 6 fractions of 9 Gy
Lymph node metastasis: 5 fractions of 6 Gy
Bone metastasis: 5 fractions of 4 Gy
   Whatever the location, radiotherapy will be administered at the rate of 2 or 3 fractions per week. The time between 2 sessions will be from 2 to 3 days.

PHASE IB: Inclusion of 5 patients by location. Each location is analysed independently.
Step 1: Bioavailability study which is performed one week before RT beginning:

First injection of AGuIX at a concentration of 50 mg/kg on D1. MRI scan is performed using MR-Linac after 2 hours, 4 hours, 3 days and 5 days to assess the intake tumour contrast.
If loss of contrast on D3 in more than one patient: study stops for the concerned location.
If loss of contrast on D5 in more than 2 patients: inclusion of 5 new patients with 5 injections of AGuIX at a concentration of 50 mg/kg

In case of loss of contrast, Dotarem or MultiHance injections may be used to visualize the tumor.
Step 2: Safety study:
   Carry out of a second injection of AGuIX on D8 followed by the 1st radiotherapy fraction (for which the schedule is established according to the results of the previous bioavailability study).
   Carry out a third injection of AGuIX on D15 before the radiotherapy fraction.
   Performing 4 to 6 radiotherapy fractions depending on the tumour location treated (D8, D10, D12, D15+/−D17+/−D19) with an MRI scan performed at each session.
If we highlight grade ≥2 toxicity in more than 2 patients: inclusion of 5 new patients with 5 injections of AGuIX at a concentration of 50 mg/kg.

### Example 3: Comparison of AGuIX and Multihance Detection at Different Concentrations by MRI of the ViewRay Linac MRI

To determine the capacity of AGuIX nanoparticles to be used as positive MRI contrast agent for

ViewRay Linac MRI displaying magnetic field of 0.35 T (MRIdian MR-linac, ViewRay Inc., Oakwood, USA) (S. Klüter, Clinical and Translational Radiation Oncology, 2019), samples with different concentrations in gadolinium were tested and compared to Multihance (Bracco) using the Torso coil (two surface flexible 6-channel coils array). The concentrations of the samples are set forth in table 1 below.

TABLE 1
Solutions of Multihance and AGulX placed in Eppendorf at
different concentrations and imaged by MRIdian from ViewRay.

| | Concentration | Concentration |
|---|---|---|
| Number of sample | multihance ([Gd³⁺]) | (AGulX/[Gd³⁺]) |
| 1 | 500 mM | 50 g · L⁻¹/50 mM |
| 2 | 250 mM | 25 g · L⁻¹/25 mM |
| 3 | 100 mM | 10 g · L⁻¹/10 mM |
| 4 | 50 mM | 5 g · L⁻¹/5 mM |
| 5 | 25 mM | 2.5 g · L⁻¹/2.5 mM |
| 6 | 10 mM | 1 g · L⁻¹/1 mM |
| 7 | 5 mM | 0.5 g · L⁻¹/0.5 mM |
| 8 | 2.5 mM | 0.25 g · L⁻¹/0.25 mM |
| 9 | 1 mM | 0.1 g · L⁻¹/0.1 mM |
| 10 | 0.5 mM | 50 mg · L⁻¹/0.05 mM |
| 11 | 0.25 mM | 25 mg · L⁻¹/0.025 mM |
| 12 | 0.1 mM | 10 mg · L⁻¹/0.01 mM |
| 13 | 0.05 mM | 5 mg · L⁻¹/0.005 mM |
| 14 | 0.025 mM | 2.5 mg · L⁻¹/0.0025 mM |
| 15 | 0.01 mM | 1 mg · L⁻¹/0.001 mM |
| 16 | 5 μM | 0.5 mg · L⁻¹/0.5 μM |
| 17 | 2.5 μM | 0.25 mg · L⁻¹/0.25 μM |
| 18 | 1 μM | 0.1 mg · L⁻¹/0.1 μM |
| 19 | 0.5 μM | 50 μg · L⁻¹/0.05 μM |
| 20 | 0.25 μM | 25 μg · L⁻¹/0.025 μM |
| 21 | 0.1 μM | 10 μg · L⁻¹/0.01 μM |

The ViewRay MRIdian system is clinically used for external radiotherapy purpose, thus it is a very appropriate device dedicated to the use of the AGuIX. A 2D coronal spin echo sequence was used: TR=400 ms, TE=20 ms, Flip Angle=90°, Bandwidth=57. Hz/Px, Matrix=512×512, Slice Thickness=3 mm, Field Of View=350×350×263 mm, Number of Averages=5. The total acquisition time was 12:30 minutes.

Solutions of multihance and AGuIX were prepared by dilution in PPI water from stock solutions of 0.5 mol·L⁻¹ and 100 g·L⁻¹ respectively 1 hour before imaging and placed in Eppendorf.

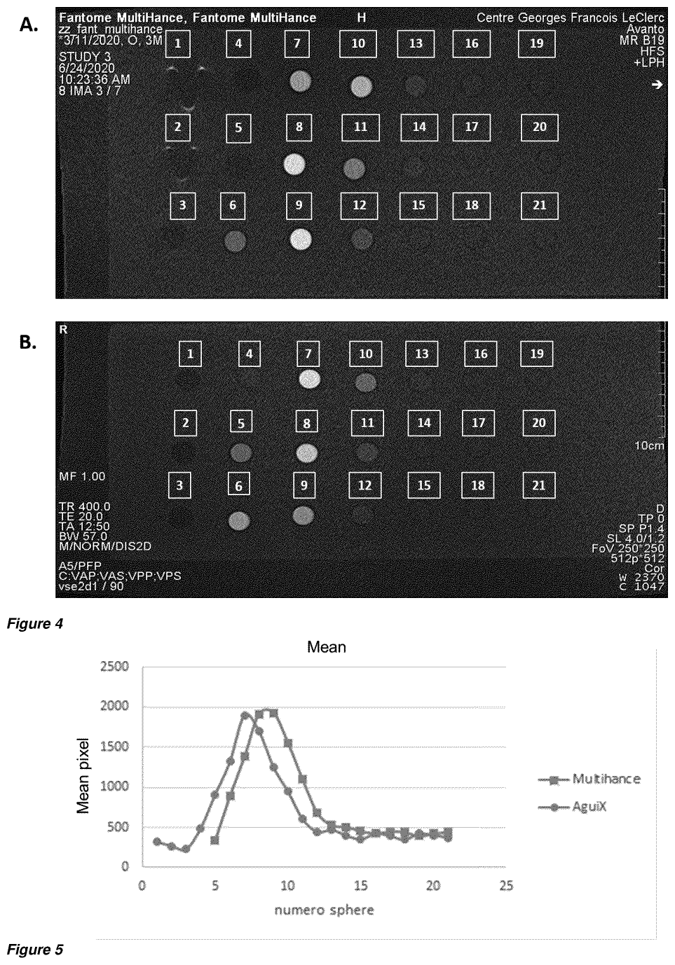

Both contrast agents can be detected at low concentrations and can act as contrast agents even at 0.35T but with a better sensitivity for AGuIX (5 μM [Gd³⁺]) in comparison with Multihance (25 μM [Gd³⁺]) as can be seen from FIGS. 4 (MRI positive signal of Multihance (A) and AGuIX (B) using MRIdian from ViewRay) and 5 (Intensity of signal for Multihance and AGuIX using MRIdian from ViewRay).

### Example 4: Comparison of AGuIX and Bi-AGuIX (50/50) Detection at Different Concentration by MRI of the ViewRay Linac MRI

Another ultrasmall nanoparticle has been tested and compared with AGuIX nanoparticles. AGuIX nanoparticles are sub 5 nm nanoparticles displaying polysiloxane matrix and covalently grafted gadolinium chelates (DOTAGA (Gd³⁺)) at their surface. By treating the AGuIX nanoparticles in acidic conditions 50% of the gadolinium are removed and then replaced by equivalent amount of bismuth to obtain particles with ratio Gd/Bi: 50/50 according to the process described in patent application WO 2018/107057.

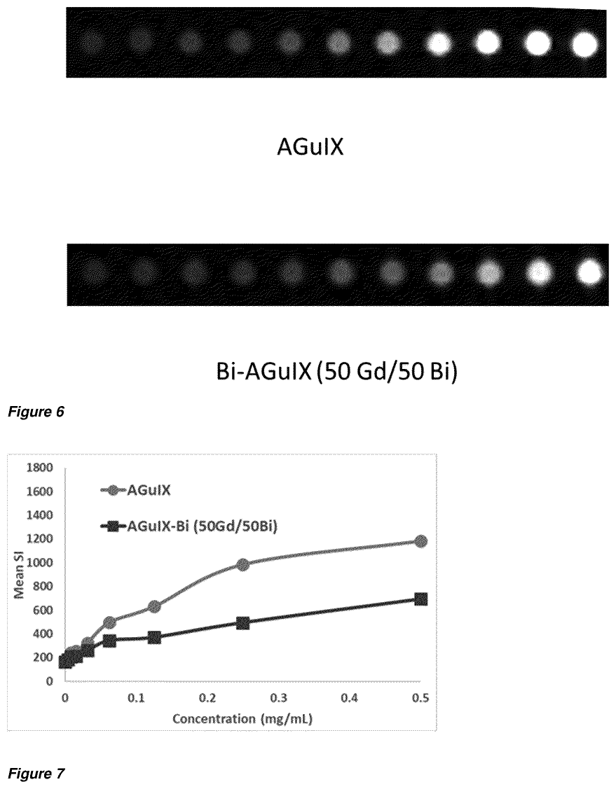

The nanoparticles are then mixed in a solution with agar agar at increasing concentrations. T1 weighted signals are then quantified by MRIdian MR-Linac (FIG. 6: Comparison of the MRI signal for AGuIX (A) and Bi-AGuIX (50/50) (B) and FIG. 7: quantification of mean signal intensity (C) using MRIdian from ViewRay). An about two-times higher signal is obtained for AGuIX in comparison with Bi-AGuIX (50/50). Even if the signal is weaker, Bi-AGuIX (50/50) are still detectable at very low concentrations in MRI emphasizing their interest for MR-Linac. The replacement of gadolinium by bismuth that displays higher atomic number (83 vs 64) will lead to higher radiosensitizing effect.

### Example 5: In Vivo Detection of Tumors by MRI of the ViewRay Linac MRI

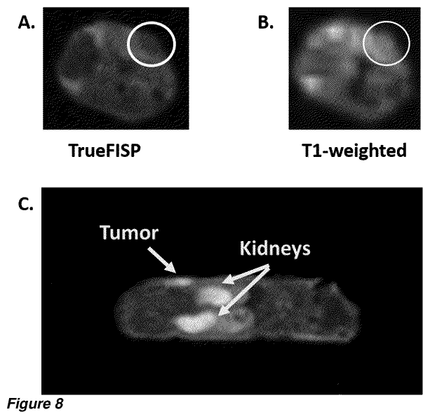

Mice with subcutaneous non-small-cell lung carcinoma (NSCLC, A549) were imaged by MRI using MRIdian MR-Linac. Two ways of administration of AGuIX were tested: intravenous and intratumoral using doses of 300 mg/kg corresponding to around 6 mg of AGuIX per mice. For both administrations, tumors can be visualized using T1-weighed sequences (FIG. 8: Imaging of subcutaneous NSCLC tumors after intravenous administration of AGuIX nanoparticles using MRIdian from ViewRay and TrueFISP (A) or T1-weighted (B) (Tumors are circled in white). Imaging of subcutaneous NSCLC tumors after intratumoral administration of AGuIX nanoparticles using MRIdian from ViewRay and TrueFISP sequence (C)). AGuIX nanoparticles are ultrasmall nanoparticles displaying size close to nm and their uptake by the kidneys can also be followed for both administrations.

1. A method of treating a tumor in a subject in need thereof, the method comprising
(i) administering an efficient amount of high-Z element containing nanoparticles having contrast enhancement for magnetic resonance imaging and/or radiosensitizing properties for radiation therapy, in a subject in need thereof, and,
(ii) exposing said subject to magnetic resonance image-guided radiation therapy by means of a Magnetic Resonance Imaging Guided Linear Accelerator (MR-Linac),
wherein said high-Z element containing nanoparticles are nanoparticles containing an element with an atomic Z number higher than 40, preferably higher than 50,
wherein said nanoparticles have a mean hydrodynamic diameter 20 nm or less, for example between 1 and 10 nm, preferably between 2 and 8 nm, and
wherein said subject is exposed to 2 or more sessions of magnetic resonance image-guided radiation therapy after a single administration of an efficient amount of said high-Z containing nanoparticles, for example between 2 and 7 sessions.

2. The method of claim 1, wherein said MR-Linac is preferably selected among MR-Linac with magnetic strength field of 0.5 T or lower strength field, for example 0.35 T.

3. The method of claim 1, wherein said nanoparticles comprise, as high-Z element, a rare earth metal, or a mixture of rare earth metals.

4. The method of claim 1, wherein said nanoparticles comprise, as high-Z element, gadolinium, bismuth, or a mixture thereof.

5. The method of claim 1, wherein said nanoparticles comprise chelates of high-Z element, for example chelates of rare earth elements.

6. The method of claim 1, wherein said nanoparticles comprise
polyorganosiloxane,
chelates covalently bound to said polyorganosiloxane,
high-Z elements complexed by the chelates.

7. The method of claim 1, wherein said nanoparticles comprise
polyorganosiloxane with a silicon weight ratio of at least 8% of the total weight of the nanoparticle, preferably between 8% and 50%,
chelates covalently bound to said polyorganosiloxane, in a proportion comprising between 5 and 100, preferably between 5 and 20 per nanoparticle, and,
high-Z elements complexed to the chelates.

8. The method of claim 1, wherein said nanoparticles comprise chelates for complexing the high-Z elements, obtained by grafting one or more of the following chelating agents on said nanoparticles: DOTA, DTPA, EDTA, EGTA, BAPTA, NOTA, DOTAGA, and DTPABA, or their mixtures.

9. The method of claim 1, wherein said nanoparticles are gadolinium-chelated polysiloxane nanoparticles of the following formula 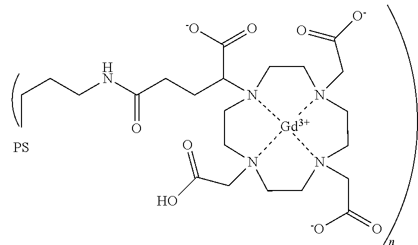
wherein PS is a matrix of polysiloxane, and,
n is comprised between 5 and 50, preferably 5 and 20, and wherein the hydrodynamic diameter is comprised between 1 and 10 nm, for example between 2 and 8 nm.

10. The method of claim 1, wherein said method comprises a first tumor pre-filling step comprising administering an effective amount of high-Z element containing nanoparticles as radiosensitizing agents in said subject in need thereof within a period between 2 and 10 days, preferably 2 and 7 days, prior to the first exposure to radiation therapy.

11. The method of claim 1, wherein said subject is exposed to at least one or more additional session of magnetic resonance image-guided radiation therapy, without further administration of a contrast agent for magnetic resonance imaging.

12. The method of claim 1, wherein said subject is exposed to 2 or more sessions of magnetic resonance image-guided radiation therapy within 5-7 days, typically with a minimum timeline of 2 or 3 days between each session.

13. The method of claim 1, wherein the subject is exposed to a dose of ionizing radiations per session of magnetic resonance image-guided radiation therapy of about 3 Gy to about 20 Gy, and the total dose is administered preferably in a maximum of 10 fractions, for example in 1 to 10 fractions, typically in 4 to 10 fractions.

14. The method of claim 1, wherein said tumor is a solid tumor, preferably selected from
(i) primary tumors of uterine cervix, rectum, lung, head and neck, prostate, colorectal, liver, and pancreas cancers and
(ii) bone metastases, typically undergoing intrafraction movements, such as sternal bones.

15. The method of claim 1, wherein said nanoparticles are administered as an injectable solution at a concentration between 50 and 150 mg/mL, and preferably between 80 and 120 mg/mL, for example 100 mg/mL, preferably by intravenous injection.

16. The method of claim 15, wherein a therapeutically effective amount administered for magnetic resonance image-guided radiation therapy is comprised between 50 mg/kg and 150 mg/kg, typically, between 80 and 120 mg/kg, for example 100 mg/kg.
